SEQUENCE LISTING (9 sequences)

SEQ ID NO: 1 - protein, 497 aa, Adeno-Associated Virus
MAADGYDWDNSGRWWDKGAKKANKDDGRGVGYKYGNGDKGVNAADAAAHDKAYDKAGDNYRYNHADARDTSGGNGRAVAKKRVGVGAKTAGKKRVSDSSSGGKTGAKKRNGTGDSSVDGATAAVGTTMASGGGAMADNNGADGVGNASGNWHCDSTWGDRVTTSTRTWATYNNHYKSSASTGASNDNHYGYSTWGYDNRHCHSRDWRNNNWGRKRNKNVKVTTNDGVTTANNTSTVVSDSYYVGSAHGCADVMYGYTNNGSAVGRSSYCYSMRTGNNTSYTVHSSYAHSSDRMNDYYYNRTNSGSANKDSRGSAGMSVKNWGCYRRVSKTKTDNNNSNTWTGASKYNNGRSNGTAMASHKDDDKMSGVMGKSAGASNTADNVMTDKATNVATRGTVAVNSSSTDATGDVHAMGAGMVWDRDVYGWAKHTDGHHSMGGGKNKNTVANASATKASTYSTGVSVWKNSKRWNVYTSNYAKSANVDTVDNNGYTRGTRYTR

SEQ ID NO: 2 - protein, 499 aa, Adeno-Associated Virus
MAADGYDWDTSGRWWKKGKARHKDDSRGVGYKYGNGDKGVNADAAAHDKAYDRDSGDNYKYNHADARKDTSGGNGRAVAKKRVGVVKTAGKKRVHSVDSSSGTGKAGARKRNGTGDADSVDGAASGGTNTMATGSGAMADNNGADGVGNSSGNWHCDSTWMGDRVTTSTRTWATYNNHYKSSSGASNDNHYGYSTWGYDNRHCHSRDWRNNNWGRKRNKNVKVTNDGTTTANNTSTVVTDSYYVGSAHGCADVMVYGYTNNGSAVGRSSYCYSMRTGNNTSYTDVHSSYAHSSDRMNDYYYSRTNTSGTTTSRSAGASDRDSRNWGCYRRVSKTSADNNNSYSWTGATKYHNGRDSVNGAMASHKDDKSGVGKGSKTNVDKVMTDRTTNVATYGSVSTNRGNRAATADVNTGVGMVWDRDVYGWAKHTDGHHSMGGGKHKNTVANSTTSAAKASTYSTGVSVWKNSKRWNYTSNYNKSVNVDTVDTNGVYSRGTRYTRN

SEQ ID NO: 3 - protein, 494 aa, Adeno-Associated Virus
MAADGYDWDNSGRWWAKGVKANHDNRRGVGYKYGGNGDKGVNADAAAHDKAYDKAGDNYKYNHADARDTSGGNGRAVAKKRGVAAKTAGKKGAVDSDSSSGVGKSGKARKRNGTGDSSVDGAATSGSNTMASGGGAMADNNGADGVGNSSGNWHCDSWGDRVTTSTRTWATYNNHYKSSSGASNDNHYGYSTWGYDNRHCHSRDWRNNNWGRKKSKNVRGVTNDGTTTANNTSTVVTDSYYVGSAHGCADVMVYGYTNNGSAVGRSSYCYSMRTGNNSYTDVHSSYAHSSDRMNDYYYNRTGTTSGTTNSRSAGSMSARNWGCYRRSKTANDNNNSNWTAASKYHNGRDSVNGAMASHKDDKMHGNGKGTTASNADNVMTDRTTNVATYGTVANNSSNTATTGTVNHGAGMVWDRDVYGWAKHTDGHHSMGGGKHMKNTVANTTSAKASTYSTGVSVWKNSKRWNYTSNYNKSVNVDTVDTNGVYSRGTRYTRN

SEQ ID NO: 4 - protein, 492 aa, Adeno-Associated Virus
MAADGYDWDNSGRWWAKGVKANHDNRRGVGYKYGGNGDKGVNADAAAHDKAYDKAGDNYKYNHADARDTSGGNGRAVAKKRGVAAKTAGKKRVDSDSSSGVGKSGKARKRNGTGDSSVDGAATSGSNTMASGGGAMADNNGADGVGNSSGNWHCDSWGDRVTTSTRTWATYNNHYKSSSGASNDNHYGYSTWGYDNRHCHSRDWRNNNWGRKKSKNVKVTNDGTTTANNTSTVVTDSYYVGSAHGCADVMVYGYTNNGSAVGRSSYCYSMRTGNNSYTDVHSSYAHSSDRMNDYYYNRTGTTSGTTNSRSAGSMSARNWGCYRRSKTANDNNNSNWTAASKYHNGRDSVNGAMASHKDDKMHGNGKGTTASNADNVMTDRTTNVATYGTVANNSSNTATTRTVNDGAGMVWDRDVYGWAKHTDGHHSMGGGKHMKNTVANTTSAKASTYSTGVSVWKNSKRWNYTSNYNKSVNVDTVDTNGVYSRGTRYTRN

SEQ ID NO: 5 - protein, 724 aa, Adeno-Associated Virus
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 6 - protein, 496 aa, Adeno-Associated Virus
MTDGYDWDNSGVRWWAGAKKANHDNARGVGYKYGGNGDKGVNAADAAAHDKAYDKAGDNYKYNHADARGDTSGGNGRAVAKKRVGVAGTAGKKRSDSSTGGKKGKAKKKVDTGAGDGGSTSGAMSDDSMRAAAGGAAVGGGADGVGNASGDWHCDSTWSGHVTTTSTRTWVTYNNHYKRGSSNTYNGSTWGYDNRHCHSRDWRNNNWGMRKAMRVKNVKVTTSNGTTVANNTSTVADSSYYVMDAGGSNDVMVYGYCGVTGNTSTDRNAYCYSMRTGNNTYSKVHSMYAHSSDRMNDYWGSTTTGTTNAGTATTNTKRTNSNKKNWGSKGSKTANNYKATGSDSKYTHSTDGRWSATGMATAGADSKSNSAGKNGNTATVGTTSAATNATDTDMWGNGGDSNSNTVDRTAGAVGMVWNRDYYGWAKHTDGHHSGGGKHKNTVANATTSSTVNSTYSTGVSVDWKRSKRWNVTSNYGNSWADAAGKYTRAGTRYTHH

SEQ ID NO: 7 - protein, 499 aa, Adeno-Associated Virus
MAADGYDWDNSGRWWDKGAKKANKDDGRGVGYKYGNGDKGVNAADAAAHDKAYDKAGDNYRYNHADARDTSGGNGRAVAKKRVGVGAKTAGKKRVSDSSSGGKTGAKKRNGTGDSSVDGATAAVGTTMASGGGAMADNNGADGVGNASGNWHCDSTWGDRVTTSTRTWATYNNHYKSSASTGASNDNHYGYSTWGYDNRHCHSRDWRNNNWGRKRNKNVKVTTNDGVTTANNTSTVVSDSYYVGSAHGCADVMYGYTNNGSAVGRSSYCYSMRTGNNTSYTDVHSSYAHSSDRMNDYYYNRTNSGSANKDSRGSAGMSVKNWGCYRRVSKTKTDNNNSNTWTGASKYNNGRSNGTAMASHKDDKDKMSGVMGKSAGASNTADNVMTDKATNVATRGTVAVNSSSTDATGDVHVMGAGMVWDRDVYGWAKHTDGHHSMGGGKHKNTVANASATKASTYSTGVSVWKNSKRWNVYTSNYAKSANVDTVDNNGYTRGTRYTR

SEQ ID NO: 8 - protein, 487 aa, Adeno-Associated Virus
MAADGYDWDNSGRWWDKGAKKANKDNGRGVGYKYGNGDKGVNAADAAAHDKAYDKAGDNYRYNHADARDTSGGNGRAVAKKRVGVGAKTAAKKRVSRSDSSTGGKKGARKRNGTGDSSVDGAASSVGSGTVAAGGGAMADNNGADGVGNASGNWHCDSTWGDRVTTSTRTWATYNNHYKSSTAGSTNDNTYGYSTWGYDNRHCHSRDWRNNNWGRKKRKNVKVTTNDGVTTANNTSTVSDSYYVGSAHGCADVMYGYTNNGSSVGRSSYCYSMRTGNNSYSDVHSSYAHSSDRMNDYYYARTSNGGTAGNRYGGSTMAAKNWGCRRVSKTDNNNSNAWTGATKYHNGRNSVNGVAMATHKDDDRSSGVGKTGATNKTTNVMTNRTNVATYGVSSNAANTAATVVNNGAGMVWNRDVYGWAKHTDGNHSMGGGKHKNTVANVTAKASTYSTGVSVWKNSKRWNYTSNKTGVDAVDSGVYSRGTRYTRN

SEQ ID NO: 9 - protein, 490 aa, Adeno-Associated Virus
MAADGYDWDNSGRWWAKGAKKANKDDGRGVGYKYGNGDKGVNAADAAAHDKAYDAGDNYRYNHADARDTSGGNGRAVAKKRVGVGAKTAGKKRVSRSDSSTGGKKGARKRNGTGDSSVDGAASGVGNTMAAGGGAMADNNGADGVGSSSGNWHCDSTWGDRVTTSTRTWATYNNHYKSNGTSGGATNDNTYGYSTWGYDNRHCHSRDWRNNNWGRKRSKNVKVTNGTKTANNTSTVTDSYYVGSAHGCADVMYGYTNNGSAVGRSSYCYSMRTGNNTYTDVHSSYAHSSDRMNDYYYSRTTTGGTANTTGSGGNTMANAKNWGCYRRVSTTTGNNNSNAWTAGTKYHNGRNSANGAMATHKDDRSNGGKNAARDNADYSDVMTSKTTNVATYGVADNNTAGTVNSGAGMVWNRDVYGWAKHTDGNHSMGGGKHKNTVADTTNSKNSTYSTGVSVWKNSKRWNYTSNYYKSTSVDAVNTGVYSRGTRYTRN

(19) United States
(12) Patent Application Publication
(10) Pub. No.: US 2023/0111672 A1
(43) Pub. Date: Apr. 13, 2023

(54) COMPOSITIONS AND METHODS FOR PRODUCTION OF RECOMBINANT ADENO-ASSOCIATED VIRUS

(71) Applicant: The Regents of the University of California, Oakland, CA (US)

(72) Inventors: David Vernon Schaffer, Berkeley, CA (US); Hyuncheol Lee, Berkeley, CA (US); Christopher Ryan Barnes, Berkeley, CA (US)

(21) Appl. No.: 17/911,306

(22) PCT Filed: Apr. 20, 2021
(86) PCT No.: PCT/US21/28153

§ 371 Date: Sep. 13, 2022

Related U.S. Application Data
(60) Provisional application No. 63/015,954, filed on Apr. 27, 2020.

(51) Int. Cl. C12N 15/86; C12N 15/69

(52) U.S. Cl. CPC C12N 15/86; C12N 15/69; C12N 2710/00052

(57) ABSTRACT

The present disclosure provides methods for producing recombinant adeno-associated virus (rAAV) virions. The present disclosure provides compositions for producing rAAV virions.

### CROSS-REFERENCE

This application claims the benefit of U.S. Provisional Patent Application No. 63/015,954, filed Apr. 27, 2020, which application is incorporated herein by reference in its entirety.

### INTRODUCTION

Gene therapy is a therapeutic modality that involves the delivery of DNA to a cell, e.g., to treat a disease. Common delivery technologies include viral vectors, lipid delivery, and naked-DNA delivery, and while the latter two technologies boast low immune profiles, repeat administration ability, and lack of transgene size limit, the technologies are highly inefficient in vivo. Viral vectors are far more efficient and include a number of properties that make them advantageous. A currently used viral vector for in vivo delivery is Adeno-Associated Virus (AAV). AAV exhibits low immunogenicity and low random integration rate, making it one of the safest DNA delivery methods. Nucleic acid encoding a gene product to be delivered can be inserted into the AAV genome, generating a recombinant AAV (rAAV).

As clinical trials using AAV have increased in number, and the understanding of AAV's natural life cycle has deepened, technologies for rAAV manufacturing have also been developed. Although various platforms, such as engineered HeLa cell systems or baculovirus production systems, are available, the most commonly used method for rAAV manufacturing involves plasmid DNA transfection into human embryonic kidney 293 (HEK293) cells. For this method, three separate plasmids—an AAV trans-plasmid comprising rep and cap genes, an AAV cis-plasmid encoding transgene (and associated regulatory elements), and a helper plasmid encoding specific adenoviral genes—are transfected into HEK293 cells. These cells, their supernatant, or both, are harvested at typically 48-96 hours post transfection with 100% confluency and subjected to rAAV purification. However, the rAAV manufacturing productivity this method offers is insufficient to meet the clinical demand for AAV gene therapy to date, such that rAAV manufacturing is a major hurdle of gene therapy.

There is a need in the art for methods for producing rAAV virions.

### SUMMARY

The present disclosure provides methods for producing recombinant adeno-associated virus (rAAV) virions. The present disclosure provides compositions for producing rAAV virions.

### BRIEF DESCRIPTION OF THE DRAWINGS

FIGS. 1A and 1B depict cell cycle arrest by thymidine treatment.

FIG. 2 is a schematic depiction of a viral manufacturing enhancement process according to the present disclosure.

FIG. 3A-3C depict rAAV titer at 72 hours with thymidine treatment.

FIG. 4 is a schematic depiction of a viral manufacturing enhancement process according to the present disclosure.

FIG. 5 depicts rAAV titer at 96 hours post transfection.

FIG. 6 depicts rAAV titer following transfection, with and without helper functions.

FIG. 7A-7C provide amino acid sequences of AAV capsid polypeptides of various AAV serotypes.

### DEFINITIONS

“AAV” is an abbreviation for adeno-associated virus, and may be used to refer to the virus itself or derivatives thereof. The term covers all subtypes and both naturally occurring and recombinant forms, except where required otherwise. The abbreviation “rAAV” refers to recombinant adeno-associated virus, also referred to as a recombinant AAV vector (or “rAAV vector”). The term “AAV” includes AAV type 1 (AAV-1), AAV type 2 (AAV-2), AAV type 3 (AAV-3), AAV type 4 (AAV-4), AAV type 5 (AAV-5), AAV type 6 (AAV-6), AAV type 7 (AAV-7), AAV type 8 (AAV-8), AAV type 9 (AAV-9), AAV type 10 (AAV-10), AAV type 11 (AAV-11), avian AAV, bovine AAV, canine AAV, equine AAV, primate AAV, non-primate AAV, and ovine AAV. See, e.g., Mori et al. (2004) Virology 330:375. The term “AAV” also includes chimeric AAV. “Primate AAV” refers to AAV isolated from a primate, “non-primate AAV” refers to AAV isolated from a non-primate mammal, “bovine AAV” refers to AAV isolated from a bovine mammal (e.g., a cow), etc.

An “rAAV vector” as used herein refers to an AAV vector comprising a polynucleotide sequence not of AAV origin (i.e., a polynucleotide heterologous to AAV), typically a sequence of interest for the genetic transformation of a cell. In general, the heterologous polynucleotide is flanked by at least one, and generally by two AAV inverted terminal repeat sequences (ITRs). The term rAAV vector encompasses both rAAV vector particles and rAAV vector plasmids.

An “AAV virus” or “AAV viral particle” or “rAAV vector particle” refers to a viral particle composed of at least one AAV capsid protein (typically by all of the capsid proteins of a wild-type AAV) and an encapsidated polynucleotide rAAV vector. If the particle comprises a heterologous polynucleotide (i.e. a polynucleotide other than a wild-type AAV genome, such as a transgene to be delivered to a mammalian cell), it is typically referred to as an “rAAV vector particle” or simply an “rAAV vector”. Thus, production of rAAV particle necessarily includes production of rAAV vector, as such a vector is contained within an rAAV particle.

“Packaging” refers to a series of intracellular events that result in the assembly and encapsidation of an AAV particle.

AAV “rep” and “cap” genes refer to polynucleotide sequences encoding replication and encapsidation proteins of adeno-associated virus. AAV rep and cap are referred to herein as AAV “packaging genes.”

A “helper virus” for AAV refers to a virus that allows AAV (e.g. wild-type AAV) to be replicated and packaged by a mammalian cell. A variety of such helper viruses for AAV are known in the art, including adenoviruses, herpesviruses and poxviruses such as vaccinia. The adenoviruses encompass a number of different subgroups, although Adenovirus type 5 of subgroup C is most commonly used. Numerous adenoviruses of human, non-human mammalian and avian origin are known and available from depositories such as the ATCC. Viruses of the herpes family include, for example, herpes simplex viruses (HSV) and Epstein-Barr viruses (EBV), as well as cytomegaloviruses (CMV) and pseudorabies viruses (PRV); which are also available from depositories such as ATCC.

“Helper virus function(s)” refers to function(s) encoded in a helper virus genome which allow AAV replication and packaging (in conjunction with other requirements for replication and packaging described herein). As described herein, “helper virus function” may be provided in a number of ways, including by providing helper virus or providing, for example, polynucleotide sequences encoding the requisite function(s) to a producer cell in trans.

An “infectious” virus or viral particle is one that comprises a polynucleotide component which it is capable of delivering into a cell for which the viral species is tropic. The term does not necessarily imply any replication capacity of the virus. As used herein, an “infectious” virus or viral particle is one that can access a target cell, can infect a target cell, and can express a heterologous nucleic acid in a target cell. Thus, “infectivity” refers to the ability of a viral particle to access a target cell, infect a target cell, and express a heterologous nucleic acid in a target cell. Infectivity can refer to in vitro infectivity or in vivo infectivity. Assays for counting infectious viral particles are described elsewhere in this disclosure and in the art. Viral infectivity can be expressed as the ratio of infectious viral particles to total viral particles. Total viral particles can be expressed as the number of viral genome (vg) copies. The ability of a viral particle to express a heterologous nucleic acid in a cell can be referred to as “transduction.” The ability of a viral particle to express a heterologous nucleic acid in a cell can be assayed using a number of techniques, including assessment of a marker gene, such as a green fluorescent protein (GFP) assay (e.g., where the virus comprises a nucleotide sequence encoding GFP), where GFP is produced in a cell infected with the viral particle and is detected and/or measured; or the measurement of a produced protein, for example by an enzyme-linked immunosorbent assay (ELISA). Viral infectivity can be expressed as the ratio of infectious viral particles to total viral particles. Methods of determining the ratio of infectious viral particle to total viral particle are known in the art. See, e.g., Grainger et al. (2005) Mol. Ther. 11:S337 (describing a TCID50 infectious titer assay); and Zolotukhin et al. (1999) Gene Ther. 6:973.

A “replication-competent” virus (e.g. a replication-competent AAV) refers to a phenotypically wild-type virus that is infectious, and is also capable of being replicated in an infected cell (i.e. in the presence of a helper virus or helper virus functions). In the case of AAV, replication competence generally requires the presence of functional AAV packaging genes. In general, rAAV vectors as described herein are replication-incompetent in mammalian cells (especially in human cells) by virtue of the lack of one or more AAV packaging genes. Typically, such rAAV vectors lack any AAV packaging gene sequences in order to minimize the possibility that replication competent AAV are generated by recombination between AAV packaging genes and an incoming rAAV vector. In many embodiments, rAAV vector preparations as described herein are those which contain few if any replication competent AAV (rcAAV, also referred to as RCA) (e.g., less than about 1 rcAAV per 10² rAAV particles, less than about 1 rcAAV per 10⁴ rAAV particles, less than about 1 rcAAV per 10⁸ rAAV particles, less than about 1 rcAAV per 10¹² rAAV particles, or no rcAAV).

The term “polynucleotide” refers to a polymeric form of nucleotides of any length, including deoxyribonucleotides or ribonucleotides, or analogs thereof. A polynucleotide may comprise modified nucleotides, such as methylated nucleotides and nucleotide analogs, and may be interrupted by non-nucleotide components. If present, modifications to the nucleotide structure may be imparted before or after assembly of the polymer. The term polynucleotide, as used herein, refers interchangeably to double- and single-stranded molecules. Unless otherwise specified or required, any embodiment of the present disclosure that is a polynucleotide encompasses both the double-stranded form and each of two complementary single-stranded forms known or predicted to make up the double-stranded form.

A polynucleotide or polypeptide has a certain percent “sequence identity” to another polynucleotide or polypeptide, meaning that, when aligned, that percentage of bases or amino acids are the same when comparing the two sequences. Sequence similarity can be determined in a number of different manners. To determine sequence identity, sequences can be aligned using the methods and computer programs, including BLAST, available over the world wide web at ncbi.nlm.nih.gov/BLAST/. Another alignment algorithm is FASTA, available in the Genetics Computing Group (GCG) package, from Madison, Wis., USA, a wholly owned subsidiary of Oxford Molecular Group, Inc. Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, Calif., USA. Alignment programs that permit gaps in the sequence can be used. The Smith-Waterman is one type of algorithm that permits gaps in sequence alignments. See Meth. Mol. Biol. 70: 173-187 (1997). Also, the GAP program using the Needleman and Wunsch alignment method can be utilized to align sequences. See J. Mol. Biol. 48: 443-453 (1970).

Of interest is the BestFit program using the local homology algorithm of Smith Waterman (Advances in Applied Mathematics 2: 482-489 (1981) to determine sequence identity. The gap generation penalty will generally range from 1 to 5, usually 2 to 4 and in many embodiments will be 3. The gap extension penalty will generally range from about 0.01 to 0.20 and in many instances will be 0.10. The program has default parameters determined by the sequences inputted to be compared. Preferably, the sequence identity is determined using the default parameters determined by the program. This program is available also from Genetics Computing Group (GCG) package, from Madison, Wis., USA.

Another program of interest is the FastDB algorithm. FastDB is described in Current Methods in Sequence Comparison and Analysis, Macromolecule Sequencing and Synthesis, Selected Methods and Applications, pp. 127-149, 1988, Alan R. Liss, Inc. Percent sequence identity is calculated by FastDB based upon the following parameters:

Mismatch Penalty: 1.00;

Gap Penalty: 1.00;

Gap Size Penalty: 0.33; and

Joining Penalty: 30.0.

A “gene” refers to a polynucleotide containing at least one open reading frame that is capable of encoding a particular protein after being transcribed and translated.

The term “guide RNA”, as used herein, refers to an RNA that comprises: i) an “activator” nucleotide sequence that binds to a CRISPR/Cas effector polypeptide (e.g., a class 2 CRISPR/Cas effector polypeptide such as a type II, type V, or type VI CRISPR/Cas effector polypeptide) and activates the CRISPR/Cas effector polypeptide; and ii) a “targeter” nucleotide sequence that comprises a nucleotide sequence that hybridizes with a target nucleic acid. The “activator” nucleotide sequence and the “targeter” nucleotide sequence can be on separate RNA molecules (e.g., a “dual-guide RNA”); or can be on the same RNA molecule (a “single-guide RNA”).

A “small interfering” or “short interfering RNA” or siRNA is a RNA duplex of nucleotides that is targeted to a gene interest (a “target gene”). An “RNA duplex” refers to the structure formed by the complementary pairing between two regions of a RNA molecule. siRNA is “targeted” to a gene in that the nucleotide sequence of the duplex portion of the siRNA is complementary to a nucleotide sequence of the targeted gene. In some embodiments, the length of the duplex of siRNAs is less than 30 nucleotides. In some embodiments, the duplex can be 29, 28, 27, 26, 25, 24, 23, 22, 21, 20, 19, 18, 17, 16, 15, 14, 13, 12, 11 or 10 nucleotides in length. In some embodiments, the length of the duplex is 19-25 nucleotides in length. The RNA duplex portion of the siRNA can be part of a hairpin structure. In addition to the duplex portion, the hairpin structure may contain a loop portion positioned between the two sequences that form the duplex. The loop can vary in length. In some embodiments the loop is 5, 6, 7, 8, 9, 10, 11, 12 or 13 nucleotides in length. The hairpin structure can also contain 3′ or 5′ overhang portions. In some cases, the overhang is a 3′ or a 5′ overhang 0, 1, 2, 3, 4 or 5 nucleotides in length.

As used herein, the term “microRNA” refers to any type of interfering RNAs, including but not limited to, endogenous microRNAs and artificial microRNAs (e.g., synthetic miRNAs). Endogenous microRNAs are small RNAs naturally encoded in the genome which are capable of modulating the productive utilization of mRNA. An artificial microRNA can be any type of RNA sequence, other than endogenous microRNA, which is capable of modulating the activity of an mRNA. A microRNA sequence can be an RNA molecule composed of any one or more of these sequences. MicroRNA (or “miRNA”) sequences have been described in publications such as Lim, et al., 2003, Genes & Development, 17, 991-1008, Lim et al., 2003, Science, 299, 1540, Lee and Ambrose, 2001, Science, 294, 862, Lau et al., 2001, Science 294, 858-861, Lagos-Quintana et al., 2002, Current Biology, 12, 735-739, Lagos-Quintana et al., 2001, Science, 294, 853-857, and Lagos-Quintana et al., 2003, RNA, 9, 175-179. Examples of microRNAs include any RNA that is a fragment of a larger RNA or is a miRNA, siRNA, stRNA, sncRNA, tncRNA, snoRNA, smRNA, shRNA, snRNA, or other small non-coding RNA. See, e.g., US Patent Applications 20050272923, 20050266552, 20050142581, and 20050075492. A “microRNA precursor” (or “pre-miRNA”) refers to a nucleic acid having a stem-loop structure with a microRNA sequence incorporated therein. A “mature microRNA” (or “mature miRNA”) includes a microRNA that has been cleaved from a microRNA precursor (a “pre-miRNA”), or that has been synthesized (e.g., synthesized in a laboratory by cell-free synthesis), and has a length of from about 19 nucleotides to about 27 nucleotides, e.g., a mature microRNA can have a length of 19 nt, 20 nt, 21 nt, 22 nt, 23 nt, 24 nt, 25 nt, 26 nt, or 27 nt. A mature microRNA can bind to a target mRNA and inhibit translation of the target mRNA.

“Recombinant,” as applied to a polynucleotide means that the polynucleotide is the product of various combinations of cloning, restriction or ligation steps, and other procedures that result in a construct that is distinct from a polynucleotide found in nature. A recombinant virus is a viral particle comprising a recombinant polynucleotide. The terms respectively include replicates of the original polynucleotide construct and progeny of the original virus construct.

A “control element” or “control sequence” is a nucleotide sequence involved in an interaction of molecules that contributes to the functional regulation of a polynucleotide, including replication, duplication, transcription, splicing, translation, or degradation of the polynucleotide. The regulation may affect the frequency, speed, or specificity of the process, and may be enhancing or inhibitory in nature. Control elements known in the art include, for example, transcriptional regulatory sequences such as promoters and enhancers. A promoter is a DNA region capable under certain conditions of binding RNA polymerase and initiating transcription of a coding region usually located downstream (in the 3′ direction) from the promoter.

“Operatively linked” or “operably linked” refers to a juxtaposition of genetic elements, wherein the elements are in a relationship permitting them to operate in the expected manner. For instance, a promoter is operatively linked to a coding region if the promoter helps initiate transcription of the coding sequence. There may be intervening residues between the promoter and coding region so long as this functional relationship is maintained.

An “expression vector” is a vector comprising a region which encodes a polypeptide of interest, and is used for effecting the expression of the protein in an intended target cell. An expression vector also comprises control elements operatively linked to the encoding region to facilitate expression of the protein in the target. The combination of control elements and a gene or genes to which they are operably linked for expression is sometimes referred to as an “expression cassette,” a large number of which are known and available in the art or can be readily constructed from components that are available in the art.

“Heterologous” means derived from a genotypically distinct entity from that of the rest of the entity to which it is being compared. For example, a polynucleotide introduced by genetic engineering techniques into a plasmid or vector derived from a different species is a heterologous polynucleotide. A promoter removed from its native coding sequence and operatively linked to a coding sequence with which it is not naturally found linked is a heterologous promoter. Thus, for example, an rAAV that includes a heterologous nucleic acid encoding a heterologous gene product is an rAAV that includes a nucleic acid not normally included in a naturally-occurring, wild-type AAV, and the encoded heterologous gene product is a gene product not normally encoded by a naturally-occurring, wild-type AAV.

The terms “genetic alteration” and “genetic modification” (and grammatical variants thereof), are used interchangeably herein to refer to a process wherein a genetic element (e.g., a polynucleotide) is introduced into a cell other than by mitosis or meiosis. The element may be heterologous to the cell, or it may be an additional copy or improved version of an element already present in the cell. Genetic alteration may be effected, for example, by transfecting a cell with a recombinant plasmid or other polynucleotide through any process known in the art, such as electroporation, calcium phosphate precipitation, or contacting with a polynucleotide-liposome complex. Genetic alteration may also be effected, for example, by transduction or infection with a DNA or RNA virus or viral vector. Generally, the genetic element is introduced into a chromosome or mini-chromosome in the cell; but any alteration that changes the phenotype and/or genotype of the cell and its progeny is included in this term.

A cell is said to be “stably” altered, transduced, genetically modified, or transformed with a genetic sequence if the sequence is available to perform its function during extended culture of the cell in vitro. Generally, such a cell is “heritably” altered (genetically modified) in that a genetic alteration is introduced which is also inheritable by progeny of the altered cell.

The terms “polypeptide,” “peptide,” and “protein” are used interchangeably herein to refer to polymers of amino acids of any length. The terms also encompass an amino acid polymer that has been modified; for example, disulfide bond formation, glycosylation, lipidation, phosphorylation, or conjugation with a labeling component. Polypeptides such as anti-angiogenic polypeptides, neuroprotective polypeptides, and the like, when discussed in the context of delivering a gene product to a mammalian subject, and compositions therefor, refer to the respective intact polypeptide, or any fragment or genetically engineered derivative thereof, which retains the desired biochemical function of the intact protein. Similarly, references to nucleic acids encoding anti-angiogenic polypeptides, nucleic acids encoding neuroprotective polypeptides, and other such nucleic acids for use in delivery of a gene product to a mammalian subject (which may be referred to as “transgenes” to be delivered to a recipient cell), include polynucleotides encoding the intact polypeptide or any fragment or genetically engineered derivative possessing the desired biochemical function.

An “isolated” plasmid, nucleic acid, vector, virus, virion, host cell, or other substance refers to a preparation of the substance devoid of at least some of the other components that may also be present where the substance or a similar substance naturally occurs or is initially prepared from. Thus, for example, an isolated substance may be prepared by using a purification technique to enrich it from a source mixture. Enrichment can be measured on an absolute basis, such as weight per volume of solution, or it can be measured in relation to a second, potentially interfering substance present in the source mixture. Increasing enrichments of the embodiments of this disclosure are increasingly more isolated. An isolated nucleic acid, vector, virus, host cell, or other substance is in some embodiments purified, e.g., from about 80% to about 90% pure, at least about 90% pure, at least about 95% pure, at least about 98% pure, or at least about 99%, or more, pure.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a gene product” includes a plurality of such gene products and reference to “the recombinant AAV (rAAV) virion” includes reference to one or more rAAV virions and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

The present disclosure provides methods for producing recombinant adeno-associated virus (rAAV) virions. The present disclosure provides compositions for producing rAAV virions.

Methods for Producing rAAV Virions

The present disclosure provides methods for producing rAAV virions. The method comprises culturing a eukaryotic cell in a culture medium, where the eukaryotic cell comprises (is genetically modified with) one or more nucleic acids comprising: a) nucleotide sequences encoding AAV rep and cap gene products; and b) a heterologous nucleotide sequence encoding one or more heterologous gene products. The culture medium comprises a cell cycle blocking agent. The cell cycle blocking agent can be present in the culture medium in a concentration of from about 1 mM to about 100 mM. Culturing the eukaryotic cells in the culture medium results in production of the rAAV virions. In some cases, the rAAV virions produced using a method of the present disclosure are infectious but not replication competent. In some cases, the rAAV virions produced using a method of the present disclosure are replication competent. In some cases, the rAAV virions produced using a method of the present disclosure are both infectious and replication competent.

The cells can be cultured in the culture medium for a period of time of from about 12 hours to 3 days, or more than 3 days. For example, the cells can be cultured in the culture medium for a period of time of from about 12 hours to about 18 hours, from about 18 hours to about 24 hours, from about 24 hours to about 36 hours, from about 36 hours to about 72 hours, from about 72 hours to about 96 hours, or more than 96 hours.

In some cases, the genetically modified eukaryotic cells (eukaryotic cells comprising one or more nucleic acids comprising: a) nucleotide sequences encoding AAV rep and cap gene products; and b) a nucleotide sequence encoding one or more heterologous gene products) are cultured in culture medium without a cell cycle blocking agent for a first period of time; the cell cycle blocking agent is added to the culture medium after the first period of time; and the cells are cultured in the culture medium, that comprises the cell cycle blocking agent, for a second period of time. The first period of time can be from 1 hour to about 48 hours, e.g., from about 1 hour to about 6 hours, from about 6 hours to about 12 hours, from about 12 hours to about 18 hours, from about 18 hours to about 24 hours, from about 24 hours to about 36 hours, or from about 36 hours to about 48 hours. The second period of time can be from about 12 hours to 3 days, or more than 3 days; for example, the second period of time can be from about 12 hours to about 18 hours, from about 18 hours to about 24 hours, from about 24 hours to about 36 hours, from about 36 hours to about 72 hours, from about 72 hours to about 96 hours, or more than 96 hours.

The amount of rAAV virions produced using a method of the present disclosure is higher than the amount of rAAV virions produced without the use of a cell cycle blocking agent. For example, a method of the present disclosure (a method for producing rAAV virions, the method comprising culturing a genetically modified eukaryotic host cell in a culture medium, wherein the eukaryotic cell is genetically modified with one or more nucleic acids comprising: a) nucleotide sequences encoding AAV rep and cap gene products; and b) a nucleotide sequence encoding one or more heterologous gene products, and where the culture medium comprises a cell cycle blocking agent in a concentration of from about 1 mM to about 100 mM) provides for production of the rAAV virions in an amount that is at least 1.5-fold higher than the amount produced when the genetically modified eukaryotic cell is cultured in a control culture medium not comprising the cell cycle blocking agent in a concentration of from about 1 mM to about 100 mM. For example, a method of the present disclosure provides for production of the rAAV virions in an amount that is at least 1.5-fold higher, at least 2.0-fold higher, at least 2.5-fold higher, or at least 3.0-fold higher, than the amount produced when the genetically modified eukaryotic cell is cultured in a control culture medium not comprising the cell cycle blocking agent in a concentration of from about 1 mM to about 100 mM. The amount of rAAV virions produced can be expressed as viral genomes per cell (vg/cell). For example, a method of the present disclosure provides for production, per cell, of the rAAV virions in an amount that is at least 1.5-fold higher, at least 2.0-fold higher, at least 2.5-fold higher, or at least 3.0-fold higher, than the amount produced, per cell when the genetically modified eukaryotic cell is cultured in a control culture medium not comprising the cell cycle blocking agent in a concentration of from about 1 mM to about 100 mM.

In some cases, a method of the present disclosure further comprises purifying the rAAV virions from the culture medium. Methods of purifying the rAAV virions from the culture medium are known in the art.

### Eukaryotic Cells

Cells that are suitable for production of rAAV virions according to a method of the present disclosure include eukaryotic cells, e.g., mammalian cells, insect cells, and the like.

Suitable mammalian cell lines include, but are not limited to, HeLa cells (e.g., American Type Culture Collection (ATCC) No. CCL-2), CHO cells (e.g., ATCC Nos. CRL9618, CCL61, CRL9096), 293 cells (e.g., ATCC No. CRL-1573), Vero cells, NIH 3T3 cells (e.g., ATCC No. CRL-1658), Huh-7 cells, BHK cells (e.g., ATCC No. CCL10), PC12 cells (ATCC No. CRL1721), COS cells, COS-7 cells (ATCC No. CRL1651), RAT1 cells, mouse L cells (ATCC No. CCLI.3), human embryonic kidney (HEK) cells (ATCC No. CRL1573), HEK 293T cells, HLHepG2 cells, and the like.

Methods and compositions provided in the present disclosure can be utilized in any insect cell that allows for the replication of AAV and which can be maintained in culture. For example, the cell line used can be from Spodoptera frugiperda, drosophila cell lines, or mosquito cell lines (e.g., Aedes albopictus-derived cell lines). In some cases, the insect cell is susceptible to baculovirus infection (e.g., Se301, SeIZD2109, SeUCR1, Sf9, Sf900+, Sf21, BTI-TN-5B1-4, MG-1, Tn368, HzAm1, Ha2302, and Hz2E5). Such cells are suitable for producing rAAV using baculovirus expression vectors (BEVs). See, e.g., U.S. Patent Publication No. 2020/0123572.

### Rep and Cap

AAV virions are composed of a 25 nm icosahedral capsid encompassing a 4.9 kb single-stranded DNA genome with two open reading frames: rep and cap. The non-structural rep gene encodes four regulatory proteins essential for viral replication, whereas cap encodes three structural proteins (VP1-3) that assemble into a 60-mer capsid shell, AAV rep and cap genes of various AAV serotypes are known in the art.

In some cases, the capsid polypeptide encoded by the cap gene is a wild-type AAV capsid polypeptide. In some cases, the capsid polypeptide encoded by the cap gene is a variant AAV capsid polypeptide that, when present in an rAAV virion, provides for greater infection of a non-permissive cell (e.g., a retinal cell; a cardiac muscle cell; a skeletal muscle cell; a lung epithelial cell; a neuron; and the like) compared to the infectivity of a control rAAV virion comprising wild-type capsid of the same rAAV serotype from which the variant AAV capsid was derived or generated. In some cases, the capsid polypeptide encoded by the cap gene is a variant AAV capsid polypeptide that, when present in an rAAV virion, provides for greater resistance to neutralizing antibody, compared to an rAAV virion comprising wild-type capsid of the same AAV serotype from which the variant AAV capsid was derived or generated. In some cases, the capsid polypeptide encoded by the cap gene is a chimeric capsid polypeptide, e.g., a capsid polypeptide that comprises portions of capsids of at least 2 different AAV serotypes, Non-limiting examples of suitable AAV capsid variants include those disclosed in U.S. Pat. Nos. 9,441,244; 9,233,131; 10,214,566; 10,046,016; 8,663,624; 9,457,103; 10,214,785; WO 2018/022905; WO 2019/006182; and US 2020/00002386.

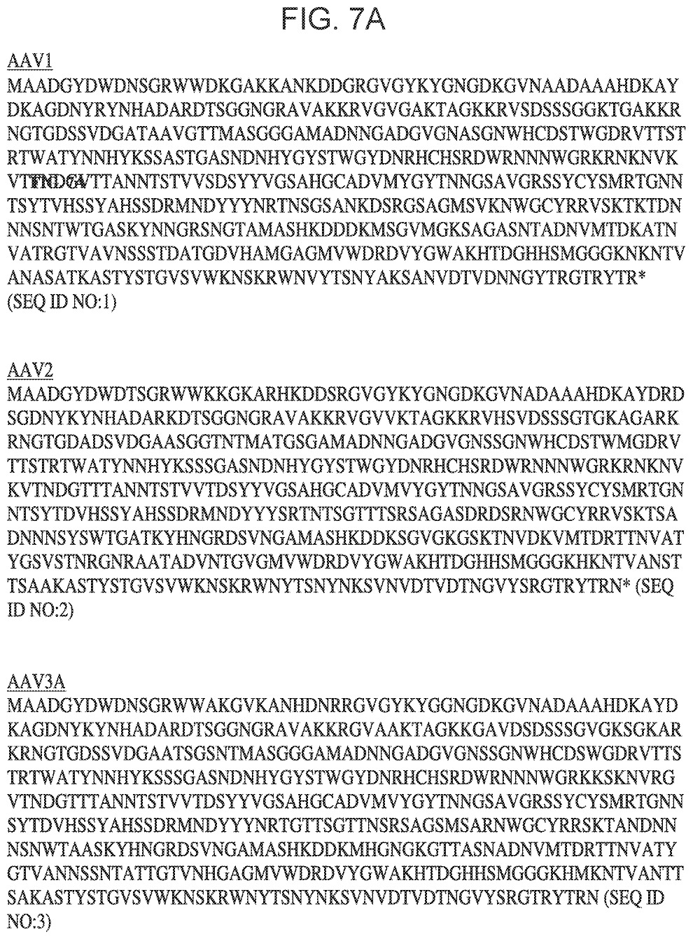

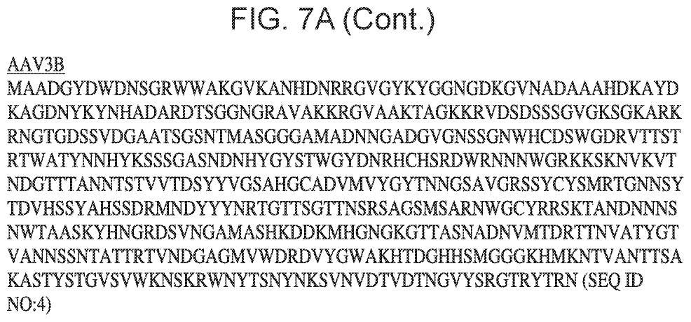

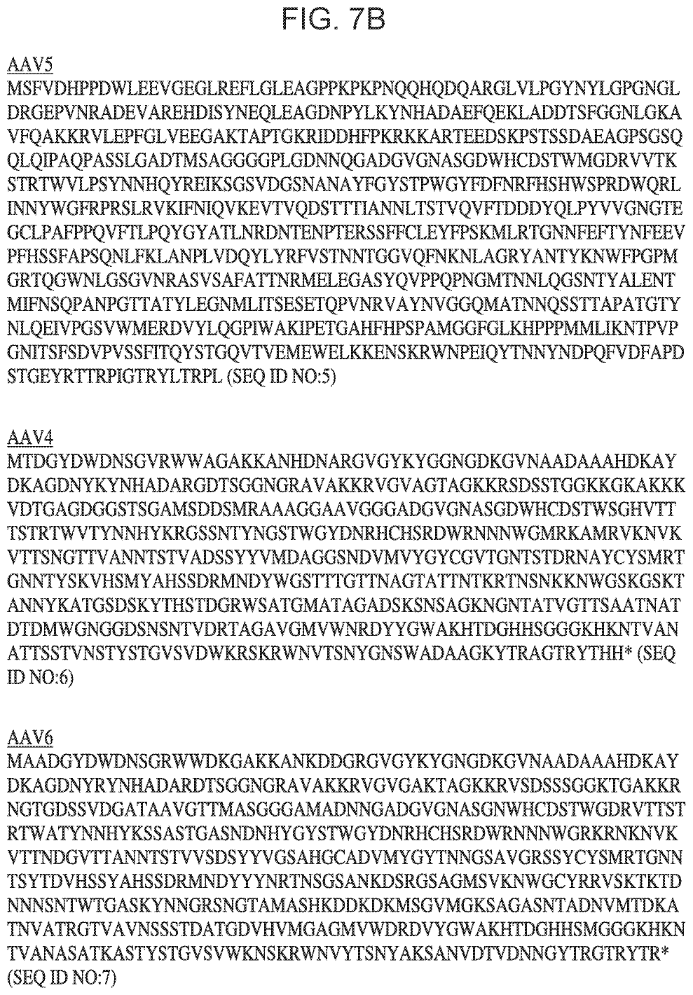

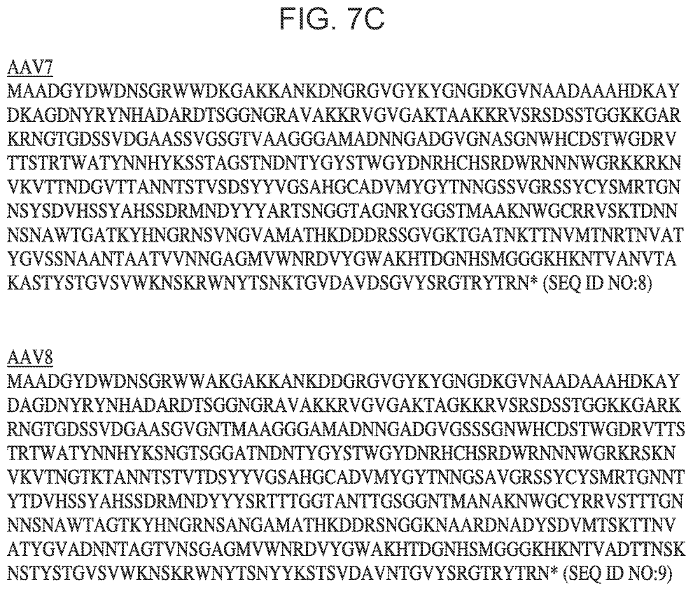

In some cases, a capsid polypeptide present in an rAAV virion produced by a method of the present disclosure comprises one or more (e.g., from 1 to 5, or from 1 to 10) amino acid substitutions compared to an AAV capsid amino acid sequence depicted in FIG. 7A-7C. In some cases, a capsid polypeptide present in an rAAV virion produced by a method of the present disclosure comprises an insertion of a heterologous peptide of from 4 amino acids to 15 amino acids in length (e.g., 4 amino acids (aa), 5 aa, 6 aa, 7 aa, 8 aa, 9 aa, 10 aa, or from 11 aa to 15 aa) in length. The heterologous peptide can be in the GH loop of the capsid.

### Agents

Any of a number of cell cycle blocking agents can be used in a method or composition of the present disclosure. The term “cell cycle blocking agent” is used herein to refer to an agent that blocks (e.g., reversibly blocks (pauses), irreversibly blocks) a cell (a eukaryotic cell) at a particular point in the cell cycle such that the cell cannot proceed further. Suitable cell cycle blocking agents include reversible cell cycle blocking agents. Reversible cell cycle blocking agents do not render the cell permanently blocked. In other words, when reversible cell cycle blocking agent is removed from the cell medium, the cell is free to proceed through the cell cycle. Cell cycle blocking agents are sometimes referred to in the art as cell synchronization agents because when such agents contact a cell population (e.g., a population having cells that are at different stages of the cell cycle), the cells of the population become blocked at the same phase of the cell cycle, thus synchronizing the population of cells relative to that particular phase of the cell cycle. When the cell cycle blocking agent used is reversible, the cells can then be “released” from cell cycle block.

Suitable cell cycle blocking agents include, but are not limited to: nocodazole (G2, M, G2/M; inhibition of microtubule polymerization), colchicine (G2, M, G2/M; inhibition of microtubule polymerization); demecolcine (colcemid) (G2, M, G2/M; inhibition of microtubule polymerization); hydroxyurea (G1, S, G1/S; inhibition of ribonucleotide reductase); aphidicolin (G1, S, G1/S; inhibition of DNA polymerase-α and DNA polymerase-δ); lovastatin (G1; inhibition of HMG-CoA reductase/cholesterol synthesis and the proteasome); mimosine (G1, S, G1/S; inhibition of thymidine, nucleotide biosynthesis, inhibition of Ctf4/chromatin binding); thymidine (G1, S, G1/S; excess thymidine-induced feedback inhibition of DNA replication); latrunculin A (M; delays anaphase onset, actin polymerization inhibitor, disrupts interpolar microtubule stability); and latrunculin B (M; actin polymerization inhibitor).

Suitable cell cycle blocking agents can include any agent that has the same or similar function as the agents above (e.g., an agent that inhibits microtubule polymerization, an agent that inhibits ribonucleotide reductase, an agent that inhibits DNA polymerase-α and/or DNA polymerase-δ, an agent that inhibits HMG-CoA reductase and/or cholesterol synthesis, an agent that inhibits nucleotide biosynthesis, an agent that inhibits DNA replication, i.e., inhibit DNA synthesis, an agent that inhibits initiation of DNA replication, an agent that inhibits deoxycytosine synthesis, an agent that induces excess thymidine-induced feedback inhibition of DNA replication, and agent that disrupts interpolar microtubule stability, an agent that inhibits actin polymerization, and the like). Suitable agents that block G1 can include: staurosporine, dimethyl sulfoxide (DMSO), glycocorticosteroids, and/or mevalonate synthesis inhibitors. Suitable agents that block G2 phase can include CDK1 inhibitors e.g., RO-3306. Suitable agents that block M can include cytochalasin D.

In some cases, suitable cell cycle blocking agents include: cobtorin; dinitroaniline; benefin (benluralin); butralin; dinitramine; ethalfluralin; oryzalin; pendimethalin; trifluralin; amiprophos-methyl; butamiphos dithiopyr; thiazopyr propyzamider-pronamide-tebutam DCPA (chlorthal-dimethyl); anisomycin; alpha amanitin; jasmonic acid; abscisic acid; menadione; cryptogeine; hydrogen peroxide; sodium permanganate; indomethacin; epoxomycin; lactacystein; icrf 193; olomoucine; roscovitine; bohemine; K252a; okadaic acid; endothal; caffeine; MG132; cycline dependent kinase inhibitors; and the like.

For more information regarding cell cycle blocking agents, see Merrill G F, Methods Cell Biol. 1998; 57:229-49, which is hereby incorporated by reference in its entirety.

In some cases, the cell cycle blocking agent is selected from the group consisting of: nocodazole, hydroxyurea; colchicine; demecolcine (colcemid); lovastatin; mimosine; thymidine; aphidicolin; latrunculin A; and latrunculin B. In some cases, the cell cycle blocking agent is thymidine.

### Heterologous Gene Products

As noted above, a method of the present disclosure comprises culturing a genetically modified eukaryotic cell in a culture medium, where the eukaryotic cell comprises (is genetically modified with) one or more nucleic acids comprising: a) nucleotide sequences encoding AAV rep and cap gene products; and b) a heterologous nucleotide sequence encoding one or more heterologous gene products. Heterologous gene products can be polypeptides or nucleic acids, or a combination of polypeptides and nucleic acids.

Suitable heterologous gene products include polypeptides, where suitable polypeptides include, but are not limited to, a neuroprotective polypeptide, an anti-angiogenic polypeptide, a growth factor, a polypeptide that provides for enhanced function of a cell, a CRISPR/Cas effector polypeptide, and the like. Suitable heterologous gene products include: a) a type II CRISPR/Cas effector polypeptide, b) a type V CRISPR/Cas effector polypeptide; c) a type VI CRISPR/Cas effector polypeptide; d) an enzymatically inactive CRISPR/Cas polypeptide; e) a nickase CRISPR/Cas effector polypeptide; and f) a CRISPR/Cas effector polypeptide and a guide RNA (e.g., a single-molecule guide RNA (a “single-guide” RNA). Suitable heterologous gene products include interfering RNAs. Suitable heterologous gene products include siRNAs. Suitable heterologous gene products include microRNAs. Suitable heterologous gene products include aptamers. Suitable heterologous gene products include fluorescent proteins (e.g., green fluorescent protein (GFP); cyan fluorescent protein; yellow fluorescent protein; red fluorescent protein; and the like).

The nucleotide sequence encoding the heterologous gene product(s) can be under the control of a promoter, e.g., a promoter that is functional in a eukaryotic cell. For expression in a eukaryotic cell, suitable promoters include, but are not limited to, light and/or heavy chain immunoglobulin gene promoter and enhancer elements; cytomegalovirus immediate early promoter; herpes simplex virus thymidine kinase promoter; early and late SV40 promoters; promoter present in long terminal repeats from a retrovirus; mouse metallothionein-I promoter; and various art-known tissue specific promoters.

Suitable reversible promoters, including reversible inducible promoters are known in the art. Suitable reversible promoters, and systems based on such reversible promoters but also comprising additional control proteins, include, but are not limited to, alcohol regulated promoters (e.g., alcohol dehydrogenase I (alcA) gene promoter, promoters responsive to alcohol transactivator proteins (AlcR), etc.), tetracycline regulated promoters, (e.g., promoter systems including TetActivators, TetON, TetOFF, etc.), steroid regulated promoters (e.g., rat glucocorticoid receptor promoter systems, human estrogen receptor promoter systems, retinoid promoter systems, thyroid promoter systems, ecdysone promoter systems, mifepristone promoter systems, etc.), metal regulated promoters (e.g., metallothionein promoter systems, etc.), pathogenesis-related regulated promoters (e.g., salicylic acid regulated promoters, ethylene regulated promoters, benzothiadiazole regulated promoters, etc.), temperature regulated promoters (e.g., heat shock inducible promoters (e.g., HSP-70, HSP-90, soybean heat shock promoter, etc.), light regulated promoters, synthetic inducible promoters, and the like.

Suitable promoters include, but are not limited to, a CAG promoter (Miyazaki et al. (1989) Gene 79:269); a cytomegalovirus (CMV) promoter; a glutamate metabotropic receptor-6 (grm6) promoter (Cronin et al. (2014) EMBO Mol. Med. 6:1175); a Pleiades promoter (Portales-Casamar et al. (2010) Proc. Natl. Acad. Sci. USA 107:16589); a choline acetyltransferase (ChAT) promoter (Misawa et al. (1992) J. Biol. Chem. 267:20392); a vesicular glutamate transporter (V-glut) promoter (Zhang et al. (2011) Brain Res. 1377:1); a glutamic acid decarboxylase (GAD) promoter (Rasmussen et al. (2007) Brain Res. 1144:19; Ritter et al. (2016) J. Gene Med. 18:27); a cholecystokinin (CCK) promoter (Ritter et al. (2016) J. Gene Med. 18:27); a parvalbumin (PV) promoter; a somatostatin (SST) promoter; a neuropeptide Y (NPY) promoter; and a vasoactive intestinal peptide (VIP) promoter. Suitable promoters include, but are not limited to, a red cone opsin promoter, rhodopsin promoter, a rhodopsin kinase promoter, and a GluR promoter (e.g., a GluR6 promoter; also referred to as grm6). Suitable promoters include, but are not limited to, a vitelliform macular dystrophy 2 (VMD2) gene promoter, and an interphotoreceptor retinoid-binding protein (IRBP) gene promoter. Also suitable for use is an L7 promoter (Oberdick et al. (1990) Science 248:223), a thy-1 promoter, a recoverin promoter (Wiechmann and Howard (2003) Curr. Eye Res. 26:25); a calbindin promoter; and a beta-actin promoter. Suitable promoters include synthetic (non-naturally occurring) promoter/enhancer combinations. In some cases, the promoter is a retinal cell-specific promoter. In some cases, the promoter is a muscle cell-specific promoter. In some cases, the promoter is a neuron-specific promoter.

In some cases, the promoter is a human synapsin (hSyn) promoter, a human elongation factor 1-α (EF1α) promoter, a cytomegalovirus (CMV) promoter, a CMV early enhancer/chicken β actin (CAG) promoter, a synapsin-I promoter (e.g., a human synapsin-I promoter), a human synuclein 1 promoter, a human Thy1 promoter, a calcium/calmodulin-dependent kinase II alpha (CAMKIIα) promoter, a vesicular γ-amino butyric acid (VGAT) promoter, a glial fibrillary acidic protein (GFAP) promoter, a Pet1 promoter, a neuropeptide Y (NPY) promoter, a somatostatin (SST) promoter, an arginine vasopressin (AVP) promoter, or a hypocretin (Hcrt) promoter.

Suitable promoters include, e.g., a CamKII promoter, a human synapsin promoter, a Thy1 promoter, a glial fibrillary acid protein (GFAP) promoter (see, e.g., Lee et al. (2008) Glia 56:481), a vesicular gamma amino butyric acid transporter (VGAT) promoter, where a PET1 promoter (see, e.g., Liu et al. (2010) Nat. Neurosci. 13:1190), a neuropeptide Y (NPY) promoter, a somatostatin (SST) promoter, an arginine vasopressin promoter (see, e.g., Pak et al. (2007) 148:3371), an Ef1a promoter, and a cytomegalovirus early enhancer/chicken β actin (CAG) promoter (see, e.g., Alexopoulou et al. (2008) MBC Cell Biol. 9:2).

Suitable promoters include a myosin light chain-2 (MLC-2) promoter, an α-myosin heavy chain (α-MHC) promoter, a desmin promoter, an AE3 promoter, a cardiac troponin C (cTnC) promoter, and a cardiac acti promoter n. Franz et al. (1997) Cardiovasc. Res. 35:560-566; Robbins et al. (1995) Ann. N.Y. Acad. Sci. 752:492-505; Linn et al. (1995) Circ. Res. 76:584-591; Parmacek et al. (1994) Mol. Cell. Biol. 14:1870-1885; Hunter et al. (1993) Hypertension 22:608-617; and Sartorelli et al. (1992) Proc. Natl. Acad. Sci. USA 89:4047-4051. See also, Pacak et al. (2008) Genet Vaccines Ther. 6:13.

### Helper Functions

In some cases, a eukaryotic cell that is used to produce an rAAV virion according to a method of the present disclosure does not include helper factors (“helper functions” or “helper polypeptides”) from any helper virus, where helper viruses include adenoviruses, herpesviruses and poxviruses such as vaccinia. In some cases, a eukaryotic cell that is used to produce an rAAV virion according to a method of the present disclosure may include a subset of the helper factors required for replication and packaging of an rAAV, but does not include the full complement of such factors.

Adenovirus helper factors include E1A, E1B, E2A, E4ORF6 and VA RNAs. Because HEK293 cells already contain the E1A/E1B gene, the helper factors that would need to be provided would be E2A, E4ORF6, and VA RNAs. In some cases, a eukaryotic cell that is used to produce an rAAV virion according to a method of the present disclosure does not include nucleic acids comprising nucleotide sequences encoding adenovirus E2A, E4ORF6, and VA RNAs. In some cases, a eukaryotic cell that is used to produce an rAAV virion according to a method of the present disclosure does not include nucleic acids comprising nucleotide sequences encoding adenovirus E1A, E1B, E2A, E4ORF6 and VA RNAs. In some cases, a eukaryotic cell that is used to produce an rAAV virion according to a method of the present disclosure does not include nucleic acids comprising nucleotide sequences encoding adenovirus E1A, E1B, E2A, and E4ORF6. In some cases, a eukaryotic cell that is used to produce an rAAV virion according to a method of the present disclosure does not include nucleic acids comprising nucleotide sequences encoding adenovirus E2A and E4ORF6.

### Compositions

The present disclosure provides a composition for the production of rAAV virions. A composition of the present disclosure comprises: a) eukaryotic cell comprising (genetically modified with) one or more nucleic acids comprising: i) nucleotide sequences encoding AAV rep and cap gene products; and ii) a nucleotide sequence encoding one or more heterologous gene products; and b) a culture medium comprising a cell cycle blocking agent in a concentration of from about 1 mM to about 100 mM.

### Eukaryotic Cells

Cells that are suitable for inclusion in a composition of the present disclosure include eukaryotic cells, e.g., mammalian cells, insect cells, and the like.

Suitable mammalian cell lines include, but are not limited to, HeLa cells (e.g., American Type Culture Collection (ATCC) No. CCL-2), CHO cells (e.g., ATCC Nos. CRL9618, CCL61, CRL9096), Vero cells, NIH 3T3 cells (e.g., ATCC No. CRL-1658), Huh-7 cells, BHK cells (e.g., ATCC No. CCL10), PC12 cells (ATCC No. CRL1721), COS cells, COS-7 cells (ATCC No. CRL1651), RAT1 cells, mouse L cells (ATCC No. CCLI.3), human embryonic kidney (HEK) cells (ATCC No. CRL1573), HEK 293T cells, HLHepG2 cells, and the like.

Suitable cells include cell lines from Spodoptera frugiperda, Drosophila cell lines, and mosquito cell lines (e.g., Aedes albopictus-derived cell lines). In some cases, the insect cell is susceptible to baculovirus infection (e.g., Se301, SeIZD2109, SeUCR1, Sf9, Sf900+, Sf21, BTI-TN-5B1-4, MG-1, Tn368, HzAm1, Ha2302, and Hz2E5). Such cells are suitable for producing rAAV using baculovirus expression vectors (BEVs). See, e.g., U.S. Patent Publication No. 2020/0123572.

### Rep and Cap

AAV virions are composed of a 25 nm icosahedral capsid encompassing a 4.9 kb single-stranded DNA genome with two open reading frames: rep and cap. The non-structural rep gene encodes four regulatory proteins essential for viral replication, whereas cap encodes three structural proteins (VP1-3) that assemble into a 60-mer capsid shell. AAV rep and cap genes of various AAV serotypes are known in the art.

In some cases, the capsid polypeptide encoded by the cap gene is a wild-type AAV capsid polypeptide. In some cases, the capsid polypeptide encoded by the cap gene is a variant AAV capsid polypeptide that, when present in an rAAV virion, provides for greater infection of a non-permissive cell (e.g., a retinal cell; a cardiac muscle cell; a skeletal muscle cell; a lung epithelial cell; a neuron; and the like) compared to the infectivity of a control rAAV virion comprising wild-type capsid of the same AAV serotype from which the variant AAV capsid was derived or generated. In some cases, the capsid polypeptide encoded by the cap gene is a variant AAV capsid polypeptide that, when present in an rAAV virion, provides for greater resistance to neutralizing antibody, compared to an rAAV virion comprising wild-type capsid of the same AAV serotype from which the variant AAV capsid was derived or generated. In some cases, the capsid polypeptide encoded by the cap gene is a chimeric capsid polypeptide, a capsid polypeptide that comprises portions of capsids of at least 2 different AAV serotypes, Non-limiting examples of suitable AAV capsid variants include those disclosed in U.S. Pat. Nos. 9,441,244; 9,233,131; 10,214,566; 10,046,016; 8,663,624; 9,457,103; 10,214,785; WO 2018/022905; WO 2019/006182; and US 2020/00002386.

In some cases, the encoded capsid polypeptide comprises one or more (e.g., from 1 to 5, or from 1 to 10) amino acid substitutions compared to an AAV capsid amino acid sequence depicted in FIG. 7A-7C, In some cases, a capsid polypeptide present in an rAAV virion produced by a method of the present disclosure comprises an insertion of a heterologous peptide of from 4 amino acids to 15 amino acids in length (e.g., 4 amino acids (aa), 5 aa, 6 aa, 7 aa, 8 an, 9 aa, 10 aa, or from 11 aa to 15 aa) in length. The heterologous peptide can be in the GH loop of the capsid.

### Agents

Any of a number of cell cycle blocking agents can be included in a composition of the present disclosure. The term “cell cycle blocking agent” is used herein to refer to an agent that blocks (e.g., reversibly blocks (pauses), irreversibly blocks) a cell (a eukaryotic cell) at a particular point in the cell cycle such that the cell cannot proceed further. Suitable cell cycle blocking agents include reversible cell cycle blocking agents. Reversible cell cycle blocking agents do not render the cell permanently blocked. In other words, when reversible cell cycle blocking agent is removed from the cell medium, the cell is free to proceed through the cell cycle. Cell cycle blocking agents are sometimes referred to in the art as cell synchronization agents because when such agents contact a cell population (e.g., a population having cells that are at different stages of the cell cycle), the cells of the population become blocked at the same phase of the cell cycle, thus synchronizing the population of cells relative to that particular phase of the cell cycle. When the cell cycle blocking agent used is reversible, the cells can then be “released” from cell cycle block.

Suitable cell cycle blocking agents include, but are not limited to: nocodazole (G2, M, G2/M; inhibition of microtubule polymerization), colchicine (G2, M, G2/M; inhibition of microtubule polymerization); demecolcine (colcemid) (G2, M, G2/M; inhibition of microtubule polymerization); hydroxyurea (G1, S, G1/S; inhibition of ribonucleotide reductase); aphidicolin (G1, S, G1/S; inhibition of DNA polymerase-α and DNA polymerase-δ); lovastatin (G1; inhibition of HMG-CoA reductase/cholesterol synthesis and the proteasome); mimosine (G1, S, G1/S; inhibition of thymidine, nucleotide biosynthesis, inhibition of Ctf4/chromatin binding); thymidine (G1, S, G1/S; excess thymidine-induced feedback inhibition of DNA replication); latrunculin A (M; delays anaphase onset, actin polymerization inhibitor, disrupts interpolar microtubule stability); and latrunculin B (M; actin polymerization inhibitor).

Suitable cell cycle blocking agents can include any agent that has the same or similar function as the agents above (e.g., an agent that inhibits microtubule polymerization, an agent that inhibits ribonucleotide reductase, an agent that inhibits DNA polymerase-α and/or DNA polymerase-δ, an agent that inhibits HMG-CoA reductase and/or cholesterol synthesis, an agent that inhibits nucleotide biosynthesis, an agent that inhibits DNA replication, i.e., inhibit DNA synthesis, an agent that inhibits initiation of DNA replication, an agent that inhibits deoxycytosine synthesis, an agent that induces excess thymidine-induced feedback inhibition of DNA replication, and agent that disrupts interpolar microtubule stability, an agent that inhibits actin polymerization, and the like). Suitable agents that block G1 can include: staurosporine, dimethyl sulfoxide (DMSO), glycocorticosteroids, and/or mevalonate synthesis inhibitors. Suitable agents that block G2 phase can include CDK1 inhibitors e.g., RO-3306. Suitable agents that block M can include cytochalasin D.

In some cases, suitable cell cycle blocking agents include: cobtorin; dinitroaniline; benefin (benluralin); butralin; dinitramine; ethalfluralin; oryzalin; pendimethalin; trifluralin; amiprophos-methyl; butamiphos dithiopyr; thiazopyr propyzamider-pronamide-tebutam DCPA (chlorthal-dimethyl); anisomycin; alpha amanitin; jasmonic acid; abscisic acid; menadione; cryptogeine; hydrogen peroxide; sodium permanganate; indomethacin; epoxomycin; lactacystein; icrf 193; olomoucine; roscovitine; bohemine; K252a; okadaic acid; endothal; caffeine; MG132; cycline dependent kinase inhibitors; and the like.

For more information regarding cell cycle blocking agents, see Merrill G F, Methods Cell Biol. 1998; 57:229-49, which is hereby incorporated by reference in its entirety.

In some cases, the cell cycle blocking agent is selected from the group consisting of: nocodazole, hydroxyurea; colchicine; demecolcine (colcemid); lovastatin; mimosine; thymidine; aphidicolin; latrunculin A; and latrunculin B. In some cases, the cell cycle blocking agent is thymidine.

### Heterologous Gene Products

As noted above, a composition of the present disclosure comprises a eukaryotic cell, where the eukaryotic cell comprises (is genetically modified with) one or more nucleic acids comprising: a) nucleotide sequences encoding AAV rep and cap gene products; and b) a heterologous nucleotide sequence encoding one or more heterologous gene products. Heterologous gene products can be polypeptides or nucleic acids, or a combination of polypeptides and nucleic acids.

Suitable heterologous gene products include polypeptides, where suitable polypeptides include, but are not limited to, a neuroprotective polypeptide, an anti-angiogenic polypeptide, a growth factor, a polypeptide that provides for enhanced function of a cell, a CRISPR/Cas effector polypeptide, and the like. Suitable heterologous gene products include: a) a type II CRISPR/Cas effector polypeptide, b) a type V CRISPR/Cas effector polypeptide; c) a type VI CRISPR/Cas effector polypeptide; d) an enzymatically inactive CRISPR/Cas polypeptide; e) a nickase CRISPR/Cas effector polypeptide; and f) a CRISPR/Cas effector polypeptide and a guide RNA (e.g., a single-molecule guide RNA (a “single-guide” RNA). Suitable heterologous gene products include interfering RNAs. Suitable heterologous gene products include interfering RNAs. Suitable heterologous gene products include siRNAs. Suitable heterologous gene products include microRNAs. Suitable heterologous gene products include aptamers. Suitable heterologous gene products include fluorescent proteins (e.g., green fluorescent protein (GFP); cyan fluorescent protein; yellow fluorescent protein; red fluorescent protein; and the like).

The nucleotide sequence encoding the heterologous gene product(s) can be under the control of a promoter, e.g., a promoter that is functional in a eukaryotic cell. For expression in a eukaryotic cell, suitable promoters include, but are not limited to, light and/or heavy chain immunoglobulin gene promoter and enhancer elements; cytomegalovirus immediate early promoter; herpes simplex virus thymidine kinase promoter; early and late SV40 promoters; promoter present in long terminal repeats from a retrovirus; mouse metallothionein-I promoter; and various art-known tissue specific promoters.

Suitable reversible promoters, including reversible inducible promoters are known in the art. Suitable reversible promoters, and systems based on such reversible promoters but also comprising additional control proteins, include, but are not limited to, alcohol regulated promoters (e.g., alcohol dehydrogenase I (alcA) gene promoter, promoters responsive to alcohol transactivator proteins (AlcR), etc.), tetracycline regulated promoters, (e.g., promoter systems including TetActivators, TetON, TetOFF, etc.), steroid regulated promoters (e.g., rat glucocorticoid receptor promoter systems, human estrogen receptor promoter systems, retinoid promoter systems, thyroid promoter systems, ecdysone promoter systems, mifepristone promoter systems, etc.), metal regulated promoters (e.g., metallothionein promoter systems, etc.), pathogenesis-related regulated promoters (e.g., salicylic acid regulated promoters, ethylene regulated promoters, benzothiadiazole regulated promoters, etc.), temperature regulated promoters (e.g., heat shock inducible promoters (e.g., HSP-70, HSP-90, soybean heat shock promoter, etc.), light regulated promoters, synthetic inducible promoters, and the like.

Suitable promoters include, but are not limited to, a CAG promoter (Miyazaki et al. (1989) Gene 79:269); a cytomegalovirus (CMV) promoter; a glutamate metabotropic receptor-6 (grm6) promoter (Cronin et al. (2014) EMBO Mol. Med. 6:1175); a Pleiades promoter (Portales-Casamar et al. (2010) Proc. Natl. Acad. Sci. USA 107:16589); a choline acetyltransferase (ChAT) promoter (Misawa et al. (1992) J. Biol. Chem. 267:20392); a vesicular glutamate transporter (V-glut) promoter (Zhang et al. (2011) Brain Res. 1377:1); a glutamic acid decarboxylase (GAD) promoter (Rasmussen et al. (2007) Brain Res. 1144:19; Ritter et al. (2016) J. Gene Med. 18:27); a cholecystokinin (CCK) promoter (Ritter et al. (2016) J. Gene Med. 18:27); a parvalbumin (PV) promoter; a somatostatin (SST) promoter; a neuropeptide Y (NPY) promoter; and a vasoactive intestinal peptide (VIP) promoter. Suitable promoters include, but are not limited to, a red cone opsin promoter, rhodopsin promoter, a rhodopsin kinase promoter, and a GluR promoter (e.g., a GluR6 promoter; also referred to as grm6). Suitable promoters include, but are not limited to, a vitelliform macular dystrophy 2 (VMD2) gene promoter, and an interphotoreceptor retinoid-binding protein (IRBP) gene promoter. Also suitable for use is an L7 promoter (Oberdick et al. (1990) Science 248:223), a thy-1 promoter, a recoverin promoter (Wiechmann and Howard (2003) Curr. Eye Res. 26:25); a calbindin promoter; and a beta-actin promoter. Suitable promoters include synthetic (non-naturally occurring) promoter/enhancer combinations. In some cases, the promoter is a retinal cell-specific promoter. In some cases, the promoter is a muscle cell-specific promoter. In some cases, the promoter is a neuron-specific promoter.

In some cases, the promoter is a human synapsin (hSyn) promoter, a human elongation factor 1-α (EF1α) promoter, a cytomegalovirus (CMV) promoter, a CMV early enhancer/chicken β actin (CAG) promoter, a synapsin-I promoter (e.g., a human synapsin-I promoter), a human synuclein 1 promoter, a human Thy1 promoter, a calcium/calmodulin-dependent kinase II alpha (CAMKIIα) promoter, a vesicular γ-amino butyric acid (VGAT) promoter, a glial fibrillary acidic protein (GFAP) promoter, a Pet1 promoter, a neuropeptide Y (NPY) promoter, a somatostatin (SST) promoter, an arginine vasopressin (AVP) promoter, or a hypocretin (Hcrt) promoter.

Suitable promoters include, e.g., a CamKII promoter, a human synapsin promoter, a Thy1 promoter, a glial fibrillary acid protein (GFAP) promoter (see, e.g., Lee et al. (2008) Glia 56:481), a vesicular gamma amino butyric acid transporter (VGAT) promoter, where a PET1 promoter (see, e.g., Liu et al. (2010) Nat. Neurosci. 13:1190), a neuropeptide Y (NPY) promoter, a somatostatin (SST) promoter, an arginine vasopressin promoter (see, e.g., Pak et al. (2007) 148:3371), an Ef1a promoter, and a cytomegalovirus early enhancer/chicken β actin (CAG) promoter (see, e.g., Alexopoulou et al. (2008) MBC Cell Biol. 9:2).

Suitable promoters include a myosin light chain-2 (MLC-2) promoter, an α-myosin heavy chain (α-MHC) promoter, a desmin promoter, an AE3 promoter, a cardiac troponin C (cTnC) promoter, and a cardiac acti promoter n. Franz et al. (1997) Cardiovasc. Res. 35:560-566; Robbins et al. (1995) Ann. N.Y. Acad. Sci. 752:492-505; Linn et al. (1995) Circ. Res. 76:584-591; Parmacek et al. (1994) Mol. Cell. Biol. 14:1870-1885; Hunter et al. (1993) Hypertension 22:608-617; and Sartorelli et al. (1992) Proc. Natl. Acad. Sci. USA 89:4047-4051. See also, Pacak et al. (2008) Genet Vaccines Ther. 6:13.

### Helper Functions

In some cases, a eukaryotic cell included in a composition of the present disclosure does not include helper factors (“helper functions” or “helper polypeptides”) from any helper virus, where helper viruses include adenoviruses, herpesviruses and poxviruses such as vaccinia. In some cases, a eukaryotic cell that is used to produce an rAAV virion according to a method of the present disclosure may include a subset of the helper factors required for replication and packaging of an rAAV, but does not include the full complement of such factors.

Adenovirus helper factors include E1A, E1B, E2A, E4ORF6 and VA RNAs. Because HEK293 cells already contain the E1A/E1B gene, the helper factors that would need to be provided would be E2A, E4ORF6, and VA RNAs. In some cases, a eukaryotic cell included in a composition of the present disclosure does not include nucleic acids comprising nucleotide sequences encoding adenovirus E2A, E4ORF6, and VA RNAs. In some cases, a eukaryotic cell included in a composition of the present disclosure does not include nucleic acids comprising nucleotide sequences encoding adenovirus E1A, E1B, E2A, E4ORF6 and VA RNAs. In some cases, a eukaryotic cell included in a composition of the present disclosure does not include nucleic acids comprising nucleotide sequences encoding adenovirus E1A, E1B, E2A, and E4ORF6. In some cases, a eukaryotic cell included in a composition of the present disclosure does not include nucleic acids comprising nucleotide sequences encoding adenovirus E2A and E4ORF6.

### Examples of Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

Aspect 1. A method for producing recombinant adeno-associated virus (rAAV) virions, the method comprising: culturing a eukaryotic cell in a culture medium, wherein the eukaryotic cell comprises one or more nucleic acids comprising: a) nucleotide sequences encoding AAV rep and cap gene products; and b) a nucleotide sequence encoding one or more heterologous gene products, wherein the culture medium comprises a cell cycle blocking agent in a concentration of from about 1 mM to about 100 mM and wherein said culturing results in production of the rAAV virions.

Aspect 2. The method of aspect 1, wherein the culture medium comprises the cell cycle blocking agent in a concentration of from about 3 mM to about 10 mM.

Aspect 3. The method of aspect 1 or aspect 2, wherein the cell cycle blocking agent is selected from nocodazole, hydroxyurea; colchicine; demecolcine (colcemid); lovastatin; mimosine; thymidine; aphidicolin; latrunculin A; and latrunculin B.

Aspect 4. The method of aspect 1 or aspect 2, wherein the cell cycle blocking agent is thymidine.

Aspect 5. The method of any one of aspects 1-4, wherein said culturing provides for production of the rAAV virions in an amount that is at least 1.5-fold higher than the amount produced when the eukaryotic cell is cultured in a control culture medium not comprising the cell cycle blocking agent in a concentration of from about 1 mM to about 100 mM.

Aspect 6. The method of any one of aspects 1-5, wherein the eukaryotic cell is a mammalian cell.

Aspect 7. The method of any one of aspects 1-5, wherein the eukaryotic cell is an insect cell.

Aspect 8. The method of any one of aspects 1-7, wherein the one or more heterologous gene products is a polypeptide.

Aspect 9. The method of any one of aspects 1-7, wherein the one or more heterologous gene products is a polynucleotide.

Aspect 10. The method of any one of aspects 1-7, wherein the one or more heterologous gene products comprise both a polypeptide and a polynucleotide.

Aspect 11. The method of any one of aspects 1-10, wherein the eukaryotic cell does not comprise a nucleic acid comprising nucleotide sequences encoding one or more adenoviral polypeptides.

Aspect 12. The method of any one of aspects 1-11, further comprising purifying the rAAV virions from the culture medium.

Aspect 13. A composition for the production of recombinant adeno-associated virus (rAAV) virions, the composition comprising: a) eukaryotic cell comprising one or more nucleic acids comprising: i) nucleotide sequences encoding AAV rep and cap gene products; and ii) a nucleotide sequence encoding one or more heterologous gene products; and b) a culture medium comprising a cell cycle blocking agent in a concentration of from about 1 mM to about 100 mM.

Aspect 14. The composition of aspect 13, wherein the culture medium comprises the cell cycle blocking agent in a concentration of from about 3 mM to about 10 mM.

Aspect 15. The composition of aspect 13 or aspect 14, wherein the cell cycle blocking agent is selected from nocodazole, hydroxyurea; colchicine; demecolcine (colcemid); lovastatin; mimosine; thymidine; aphidicolin; latrunculin A; and latrunculin B.

Aspect 16. The composition of aspect 13 or aspect 14, wherein the cell cycle blocking agent is thymidine.

Aspect 17. The composition of any one of aspects 13-16, wherein the one or more heterologous gene products is a polypeptide.

Aspect 18. The composition of any one of aspects 13-16, wherein the one or more heterologous gene products is a polynucleotide.

Aspect 19. The composition of any one of aspects 13-16, wherein the one or more heterologous gene products comprise both a polypeptide and a polynucleotide.

Aspect 20. The composition of any one of aspects 13-19, wherein the eukaryotic cell does not comprise a nucleic acid comprising nucleotide sequences encoding one or more adenoviral polypeptides.

Aspect 21. The composition of any one of aspects 13-20, wherein the eukaryotic is a mammalian cell.

Aspect 22. The composition of any one of aspects 13-20, wherein the eukaryotic is an insect cell.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1

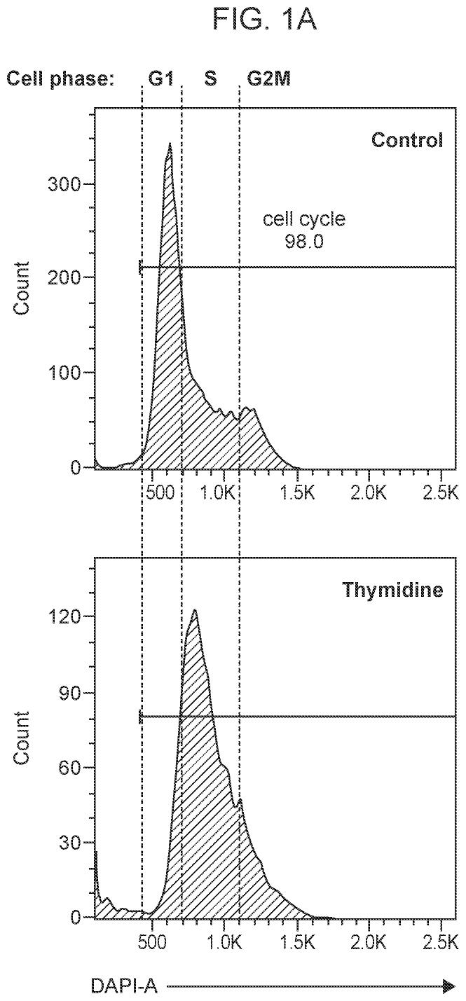

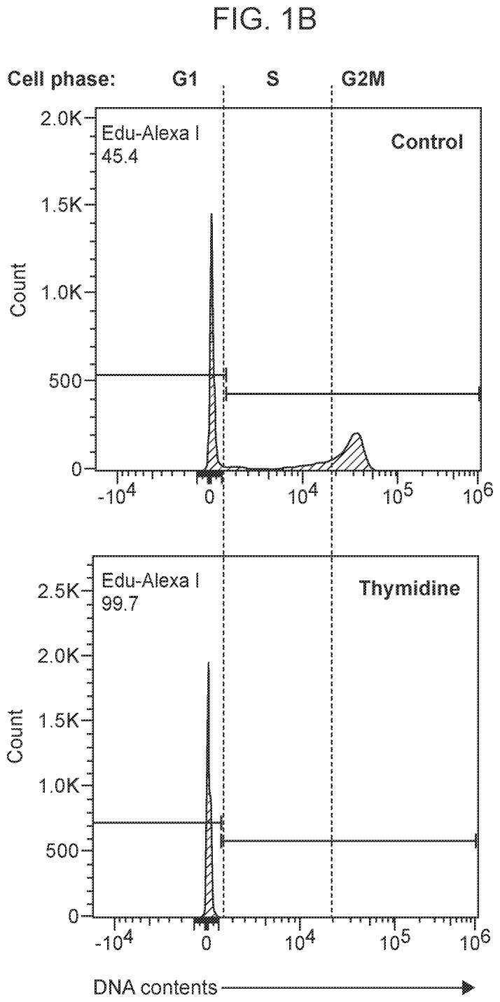

HEK 293T cells were treated with thymidine and analyzed using flow cytometry with DAPI (4′,6-diamidino-2-phenylindole) or EdU (5-ethynyl-2′-deoxyuridine) incorporation at 24 hrs post treatment. Total DNA stained by DAPI shows the cell population resides predominantly in the S-phase section (FIG. 1A), and EdU stain shows no incorporation has occurred (FIG. 1B), which implies the concentration of 4 mM thymidine is appropriate for S phase arrest.

FIG. 1A-1B. Verification of cell cycle arrest by thymidine treatment. HEK293T cells were split into 10 cm′ cell culture dishes at 3.0e6 cells/plate. Thymidine stock was prepared with molecular biology grade distilled water at a concentration of 200 mM. The final concentration of thymidine in the culture medium was 4 mM. Two hrs before harvest, cells were incubated with EdU and were harvested for flow cytometry. DAPI staining and EdU assay were performed in accordance with the manufacturer's protocol.

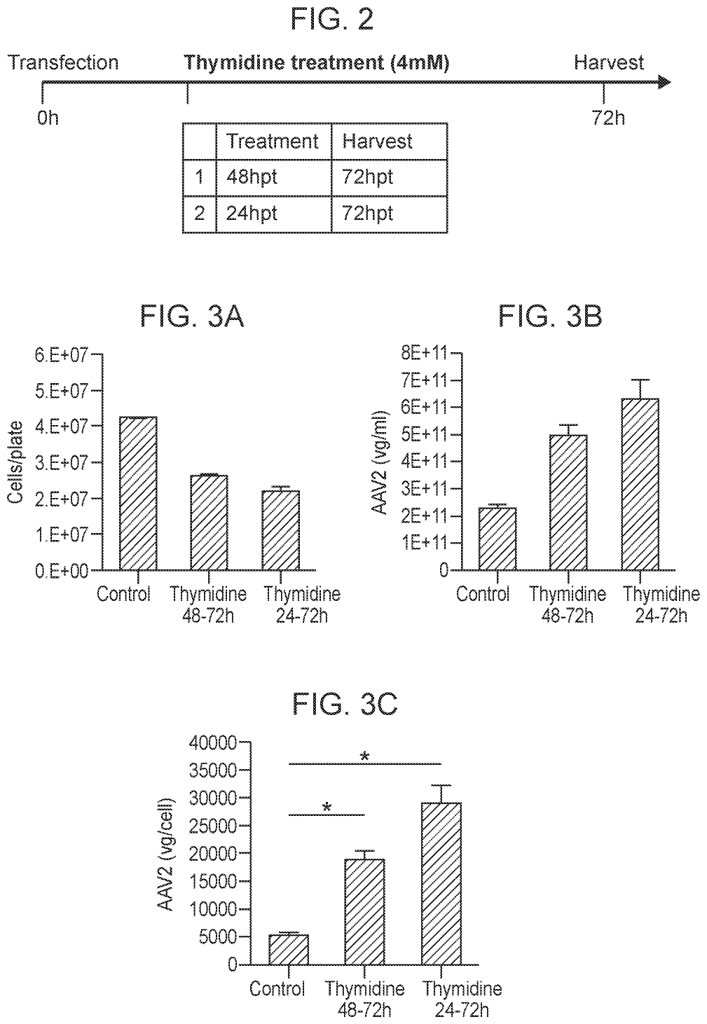

The effect of thymidine treatment for rAAV packaging was assessed with the following description. rAAV manufacturing in HEK293T cells was achieved by transfecting cells with the transgene (ss-CAG-eGFP), the adenoviral helper plasmid pHelper, and rAAV2(Rep/Cap) helper plasmid in accordance with the standard protocol of PEI (polyethyleneimine) transfection (Ojala et al. 2018). Thymidine was added onto media without media change at 24 or 48 hrs post transfection and then cell pellets were harvested at 72 hrs post transfection (FIG. 2).

FIG. 2. Schematic of the viral manufacturing enhancement process with thymidine in HEK293T cells.

The number of total cells was counted for the evaluation of cell growth prior to harvesting the cell pellet (FIG. 3A). The thymidine treated group showed lower cell count than the control group, and the count number was used for the normalization after assessment of rAAV titer. Next, cell lysate samples were incubated with Benzonase as well as DNase to remove residual plasmid, and rAAV titer with or without thymidine treatment was determined by quantitative polymerase chain reaction (qPCR) with specific primers for the transgene, eGFP (FIG. 3B). rAAV titer was then normalized to total cell count to compare viral titers in vg/cell (viral genomes/cell) (FIG. 3C).

FIG. 3A-3C. rAAV titer at 72 h with thymidine treatment. HEK293T cells were split into 10 cm′ cell culture dishes at 3.0×10⁶ cells/plate and transfected with the plasmids for rAAV packaging. Cell pellets at 72 hrs post transfection were lysed by freezing and thawing and then incubated with Benzonase, followed by DNase. Subsequent Proteinase K digestion released AAV genome ssDNA from the AAV vector, which were subjected into qPCR for measuring rAAV genome copy number. Error bars, mean±SD. *P<0.05, (All statistics are the result of a student's two tailed t-test with paired variance).

The qPCR results show that rAAV was packaged and that the treatment of thymidine enhances the rAAV titer compared to control. Thymidine treatment 48-72h shows 2.19 fold-higher titer in vg/ml while 3.52-fold higher titer in vg/cell and the treatment 24-72h shows 2.77 fold-higher in vg/ml while 5.38-fold higher than that on control cells.

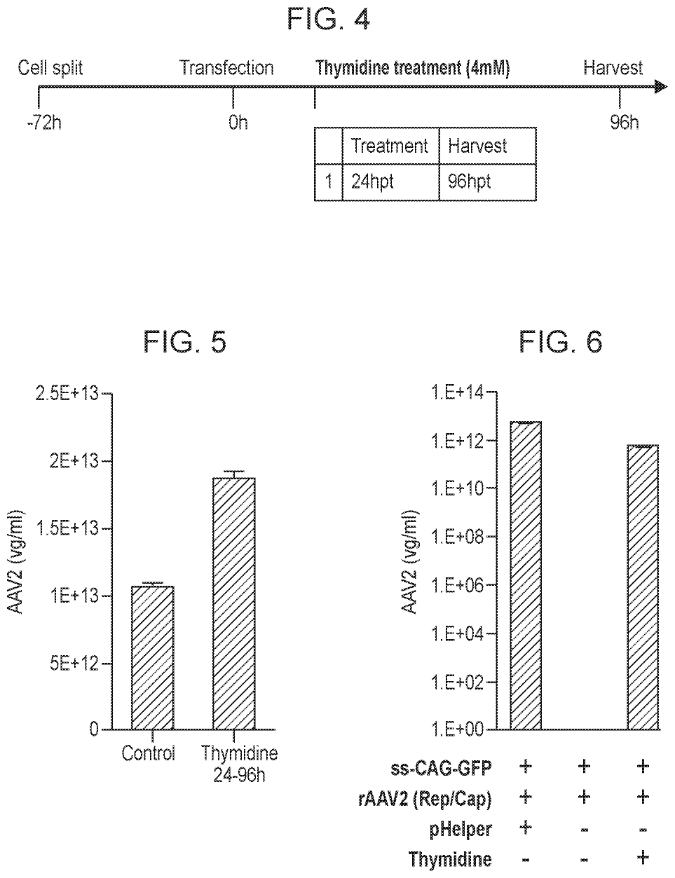

To assess the generality of the result, HEK293 cells were tested, using a different packaging method than that used for HEK293T cells. HEK293 cells were seeded at high concentration and incubated for 72 hrs to reach 100% confluency and then transfected with plasmids. Thymidine was added 24 hrs post transfection, and pellets were harvested at 96 hrs post transfection (FIG. 4).

FIG. 4. Schematic of the viral manufacturing enhancement process with thymidine in HEK293 cells.

Thymidine treated HEK293 cells show 1.75-fold higher rAAV titer in vg/ml than that of control cells as assessed by qPCR, demonstrating that thymidine treatment enhances rAAV packaging in HEK293 cells (FIG. 5).

FIG. 5. HEK293 Cells were seeded at 1.0×10⁵ cell/cm′ into 10 cm′ cell culture dish and transfected with the plasmids for rAAV packaging. Thymidine was treated at 24 hrs post transfection and cell pellets were harvested at 96 hrs post transfection. rAAV titer was measured by qPCR in the same method of FIG. 3. Error bars, mean±SD.

Plasmids containing adenoviral genes that promote AAV replication and vector production, such as pHelper, have previously been essential for rAAV packaging. That is, AAV requires co-infection of helper virus or introduction of a DNA-damaging agent to complete life cycle (Yakobson, Koch, and Winocour 1987; Yalklnoglu et al. 1988), functions that in rAAV manufacturing are typically replaced by transfection with a plasmid such as pHelper. The present disclosure provides the use of thymidine as a replacement for pHelper. The positive control cells were transfected with the three plasmids (AAV vector, AAV helper, pHelper) while the negative control cells were transfected without pHelper. In some samples, thymidine was added at 24 hrs post transfection (FIG. 6). Measuring rAAV titer below shows rAAV was packaged in the absence of pHelper only when the thymidine was added, but not in its absence.

FIG. 6. HEK293T cells were transfected with plasmids in accordance with the indication and then thymidine was added at 24 hrs post transfection. All cells were harvested at 72 hrs post transfection and rAAV titer were determined. Error bars, mean±SD.

### REFERENCES

Amberger, Joanna S., Carol A. Bocchini, Alan F. Scott, and Ada Hamosh. 2019. “OMIM.org: Leveraging Knowledge across Phenotype-Gene Relationships.” Nucleic Acids Research 47(D1): D1038-43.
Anguela, Xavier M., and Katherine A. High. 2019. “Entering the Modern Era of Gene Therapy.” Annual Review of Medicine 70(1): 273-88.
Ayuso, Eduard. 2016. “Manufacturing of Recombinant Adeno-Associated Viral Vectors: New Technologies Are Welcome.” Molecular Therapy—Methods and Clinical Development 3(October 2015): 15049. http://dx.doi.org/10.1038/mtm.2015.49.
Banfalvi, Gaspar. 2011. 761 Methods in molecular biology (Clifton, N.J.) Cell Cycle Synchronization Methods and Protocols.
Bolderson, Emma et al. 2004. “ATM Is Required for the Cellular Response to Thymidine Induced Replication Fork Stress.” Human Molecular Genetics 13(23): 2937-45.
Goswami, Reena et al. 2019. “Gene Therapy Leaves a Vicious Cycle.” Frontiers in Oncology 9(APR): 1-25.
Gruntman, Alisha M., and Terence R. Flotte. 2018. “The Rapidly Evolving State of Gene Therapy.” FASEB Journal 32(4): 1733-40.
Iourov, Ivan Y., Svetlana G. Vorsanova, and Yuri B. Yurov. 2019. “Pathway-Based Classification of Genetic Diseases.” Molecular Cytogenetics 12(1): 1-5.
O'Dwyer, Peter J., Susan A. Kins, Daniel F. Hoth, and Brian Leyland-Jones. 1987. “Role of Thymidine in Biochemical Modulation: A Review.” Cancer Research 47(15): 3911-19.
Ogden, Pierce J., Eric D. Kelsic, Sam Sinai, and George M. Church. 2019. “Comprehensive AAV Capsid Fitness Landscape Reveals a Viral Gene and Enables Machine-Guided Design.” Science 366(6469): 1139-43.
Ojala, David S. et al. 2018. “In Vivo Selection of a Computationally Designed SCHEMA AAV Library Yields a Novel Variant for Infection of Adult Neural Stem Cells in the SVZ.” Molecular Therapy 26(1): 304-19. https://doi.org/10.1016/j.ymthe.2017.09.006.
Sonntag, F. et al. 2011. “The Assembly-Activating Protein Promotes Capsid Assembly of Different Adeno-Associated Virus Serotypes.” Journal of Virology 85(23): 12686-97.
Sonntag, Florian, Kristin Schmidt, and Jurgen A. Kleinschmidt. 2010. “A Viral Assembly Factor Promotes AAV2 Capsid Formation in the Nucleolus.” Proceedings of the National Academy of Sciences of the United States of America 107(22): 10220-25.
Thul, Peter J., and Cecilia Lindskog. 2018. “The Human Protein Atlas: A Spatial Map of the Human Proteome.” Protein Science 27(1): 233-44.
Wang, Dan, Phillip W. L. Tai, and Guangping Gao. 2019. “Adeno-Associated Virus Vector as a Platform for Gene Therapy Delivery.” Nature Reviews Drug Discovery 18(5): 358-78. http://dx.doi.org/10.1038/s41573-019-0012-9.
Yakobson, B, T Koch, and E Winocour. 1987. “Replication of Adeno-Associated Virus in Synchronized Cells without the Addition of a Helper Virus.” Journal of Virology 61(4): 972-81.
Yalklnoglu, A. Özkan et al. 1988. “Dna Amplification of Adeno-Associated Virus as a Response to Cellular Genotoxic Stress.” Cancer Research 48(11): 3123-29.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A method for producing recombinant adeno-associated virus (rAAV) virions, the method comprising:
culturing a eukaryotic cell in a culture medium, wherein the eukaryotic cell comprises one or more nucleic acids comprising:
a) nucleotide sequences encoding AAV rep and cap gene products; and
b) a nucleotide sequence encoding one or more heterologous gene products,
wherein the culture medium comprises a cell cycle blocking agent in a concentration of from about 1 mM to about 100 mM and
wherein said culturing results in production of the rAAV virions.

2. The method of claim 1, wherein the culture medium comprises the cell cycle blocking agent in a concentration of from about 3 mM to about 10 mM.

3. The method of claim 1 or claim 2, wherein the cell cycle blocking agent is selected from nocodazole, hydroxyurea; colchicine; demecolcine (colcemid); lovastatin; mimosine; thymidine; aphidicolin; latrunculin A; and latrunculin B.

4. The method of claim 1 or claim 2, wherein the cell cycle blocking agent is thymidine.

5. The method of any one of claims 1-4, wherein said culturing provides for production of the rAAV virions in an amount that is at least 1.5-fold higher than the amount produced when the eukaryotic cell is cultured in a control culture medium not comprising the cell cycle blocking agent in a concentration of from about 1 mM to about 100 mM.

6. The method of any one of claims 1-5, wherein the eukaryotic cell is a mammalian cell.

7. The method of any one of claims 1-5, wherein the eukaryotic cell is an insect cell.

8. The method of any one of claims 1-7, wherein the one or more heterologous gene products is a polypeptide.

9. The method of any one of claims 1-7, wherein the one or more heterologous gene products is a polynucleotide.

10. The method of any one of claims 1-7, wherein the one or more heterologous gene products comprise both a polypeptide and a polynucleotide.

11. The method of any one of claims 1-10, wherein the eukaryotic cell does not comprise a nucleic acid comprising nucleotide sequences encoding one or more adenoviral polypeptides.

12. The method of any one of claims 1-11, further comprising purifying the rAAV virions from the culture medium.

13. A composition for the production of recombinant adeno-associated virus (rAAV) virions, the composition comprising:
a) eukaryotic cell comprising one or more nucleic acids comprising:
i) nucleotide sequences encoding AAV rep and cap gene products; and
ii) a nucleotide sequence encoding one or more heterologous gene products; and
b) a culture medium comprising a cell cycle blocking agent in a concentration of from about 1 mM to about 100 mM.

14. The composition of claim 13, wherein the culture medium comprises the cell cycle blocking agent in a concentration of from about 3 mM to about 10 mM.

15. The composition of claim 13 or claim 14, wherein the cell cycle blocking agent is selected from nocodazole, hydroxyurea; colchicine; demecolcine (colcemid); lovastatin; mimosine; thymidine; aphidicolin; latrunculin A; and latrunculin B.

16. The composition of claim 13 or claim 14, wherein the cell cycle blocking agent is thymidine.

17. The composition of any one of claims 13-16, wherein the one or more heterologous gene products is a polypeptide.

18. The composition of any one of claims 13-16, wherein the one or more heterologous gene products is a polynucleotide.

19. The composition of any one of claims 13-16, wherein the one or more heterologous gene products comprise both a polypeptide and a polynucleotide.

20. The composition of any one of claims 13-19, wherein the eukaryotic cell does not comprise a nucleic acid comprising nucleotide sequences encoding one or more adenoviral polypeptides.

21. The composition of any one of claims 13-20, wherein the eukaryotic is a mammalian cell.

22. The composition of any one of claims 13-20, wherein the eukaryotic is an insect cell.
